(19) United States
(12) Patent Application Publication
(10) Pub. No.: US 2011/0137345 A1
(43) Pub. Date: Jun. 9, 2011

(54) Posterior lumbar fusion

(76) Inventors: Caleb Stoll, Westminster, CO (US); Gregory Causey, Louisville, CO (US); Andrew Lamborne, Golden, CO (US); Michael Fulton, Superior, CO (US); Jeff Thramann, Longmont, CO (US); Lawrence Binder, Broomfield, CO (US)

(21) Appl. No.: 12/661,549

(22) Filed: Mar. 18, 2010

Related U.S. Application Data
(60) Provisional application No. 61/161,347, filed on Mar. 18, 2009.

(51) Int. Cl. A61B 17/70; A61B 17/88

(52) U.S. Cl. 606/251; 606/250; 606/263; 606/279

(57) ABSTRACT

There is disclosed an interspinous attachment system for providing support between adjacent spinal levels. In an embodiment, an interspinous attachment system provides support between adjacent spinal levels and includes a crosslink, a first crosslink connector and a second crosslink connector each selectively attachable to spinal fixation rods, and an interspinous attachment device having a crosslink attachment portion and a spinous process attachment portion. In another embodiment, there is provided a method of attaching a crosslink to a pair of spinal fixation rods. The method includes tightening a set screw in a housing of each of the crosslink connectors to engage a crosslink, rotate the crosslink connectors with the set screw tightening, and engage the rods with the cross link through the rotation, wherein the tightening the set screw fixedly connects the ends of the crosslink and the rods together.

### REFERENCE TO PENDING PRIOR PATENT APPLICATION

This application claims the benefit under 35 U.S.C. 119 (e) of U.S. Provisional Patent Application No. 61/161,347, filed Mar. 18, 2009 by Lawrence Binder, et al. for “POSTERIOR LUMBAR FUSION,” which patent application is hereby incorporated herein by reference.

### BACKGROUND

Generally, posterior lumbar fusion with a total decompression including removal of the spinous process does not readily allow insertion of a currently available spinous process spacer. This is due to the requirement of a superior spinous process and an inferior spinous process for proper insertion of the currently available spinous process spacers.

### SUMMARY

In an embodiment, there is provided an interspinous attachment system for providing support between adjacent spinal levels, the system comprising a crosslink having a first end and a second end in opposition to one another, and a distance between the first end and the second end corresponding to a distance between two spinal fixation rods fixed adjacent a spinal column of a patient; a first crosslink connector and a second crosslink connector, at least one of the first and second crosslink connectors having a housing with a first end and a second end in opposition to one another, the first end having a selectively attachable portion to one of the spinal fixation rods, the second end having a selectively attachable portion to one of the first and second ends of the crosslink; and an interspinous attachment device having a crosslink attachment portion and a spinous process attachment portion.

In another embodiment, there is provided a method of attaching a crosslink to a pair of spinal fixation rods, the method comprising providing a pair of crosslink connectors, each one of the pair of crosslink connectors having a housing with a first end and a second end in opposition to one another, the first end having a selectively attachable portion to one of the spinal fixation rods, and the second end having a selectively attachable portion to one of the first end and the second end of the crosslink; positioning the first end and the second end of the crosslink into the second end of the crosslink connectors; positioning the first end of each of the crosslink connectors onto one of the spinal fixation rods; and tightening a set screw in the housing of each of the crosslink connectors to engage the first end and the second end of the crosslink in the second end of the crosslink connectors, rotate the first end of the crosslink connectors with the set screw tightening, and engage the rods with the first end of the cross link through the rotation, wherein the tightening the set screw fixedly connects both the first end and the second end of the crosslink and the rods to the housing.

Other embodiments are also disclosed.

### BRIEF DESCRIPTION OF THE DRAWINGS

Illustrative embodiments of the invention are illustrated in the drawings, in which:

FIG. 1 illustrates a portion of a spinal column having three levels interconnected to one another;

FIG. 2 illustrates an embodiment of an interspinous attachment system providing support between adjacent spinal levels;

FIG. 3 illustrates a crosslink with a fixed length;

FIG. 4 illustrates a crosslink with a variable length;

FIGS. 5 and 6 illustrate an embodiment of crosslink connectors with a first set screw to selectively retain one of the spinal fixation rods and a second set screw to selectively retain an end of a crosslink;

FIG. 7 illustrates an embodiment of an interspinous attachment device with a band to provide support;

FIG. 8 illustrates another embodiment of an interspinous attachment device with a generally linear spinous process support;

FIGS. 9-13A illustrate various exemplary embodiments of interspinous attachment devices;

FIG. 14 illustrates crosslink connectors in direct attachment to pedicle screws and spinal fixation rods;

FIGS. 15 and 16 illustrate exemplary embodiments of interspinous attachment devices with a crosslink attachment portion and a spinous process attachment portion;

FIGS. 17A-17D and 18A-18C illustrate an embodiment of a crosslink connector with a single set screw for locking both a spinal fixation rod and an end of a crosslink together;

FIGS. 19A-19C illustrate another embodiment of a crosslink connector with a swing arm;

FIGS. 20A and 208 illustrate an embodiment of a crosslink connector with a swing arm to hold a rod in compression;

FIGS. 21A and 21B illustrate an embodiment of a crosslink connector with a swing arm and a pivot to hold a rod in compression;

FIGS. 22A-22D illustrate an embodiment of a crosslink connector with a swing arm disposed in a housing between an end of a crosslink and a rod;

FIG. 23 illustrates an embodiment of a crosslink connector with a swing arm having a slot for lateral movement with respect to a pin;

FIG. 24 illustrates an embodiment of a crosslink connector with a wedge disposed in a housing between an end of a crosslink and a rod;

FIGS. 25-29 illustrate various tools for use with interspinous attachment modular components;

FIG. 30 illustrates a crosslink in attachment to spinal fixation rods with crosslink connectors;

FIG. 31 illustrates an exemplary embodiment of a spinous process attachment portion;

FIG. 32 illustrates a cable tensioner in engagement with an interspinous attachment device;

FIGS. 33-36 illustrate an embodiment of an interspinous attachment device; and

FIG. 37 illustrates another embodiment of an interspinous attachment device.

### DETAILED DESCRIPTION

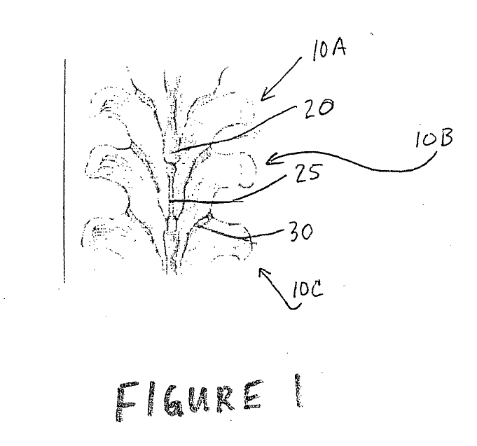

The spinous process, and the ligaments that attach to it, play an important role in motion restriction. Referring to FIG. 1, for example, there is shown a portion of a spinal column 5 having three levels 10A, 10B, and 100 interconnected to one another. A supraspinous ligament 20, an interspinous ligament 25, and ligamentum flavum 30 provide support to three levels 10A, 10B, and 100 interconnected to one another. Replacing ligament function using a surgical procedure helps bring affected spinal segments closer to normal motion.

In various embodiments, devices are provided as an adjunct to traditional posterior fusion. Adjacent level “soft” stabilization is a widely studied concept and has been accepted by many physicians for preserving a range of motion as well as providing load sharing to protect adjacent spinal anatomy (e.g., discs and facets). A band of material may also be included in a device to replace ligaments.

Attachment of a soft stabilization spinous process device, made of a suitable material, to something other than the pedicles may achieve more favorable clinical results, allow a smaller surgical incision, and reduce operating room time. For example, a load sharing device may be provided for spinal levels adjacent to a fusion. Existing rod and screw constraints may anchor the load sharing device rather than additional pedicle screws inserted at the supported level.

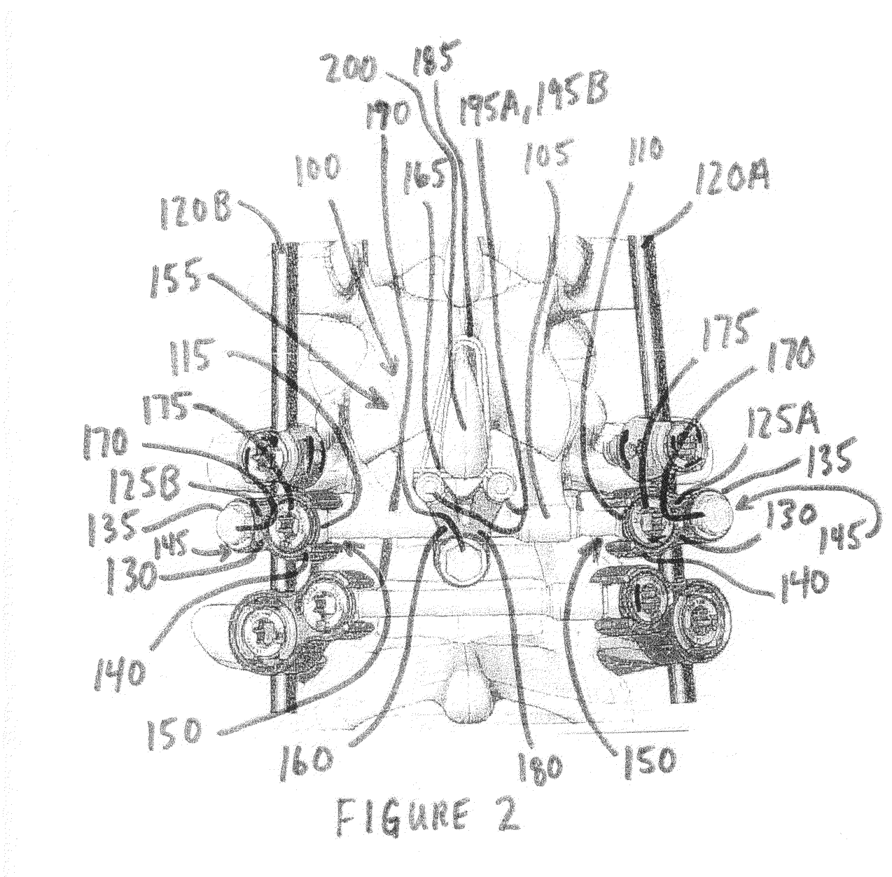

In one embodiment, and referring generally to FIG. 2, an interspinous attachment system 100 may provide support between adjacent spinal levels. System 100 may include a crosslink 105 and an interspinous attachment device 155 for coupling between crosslink 105 and a spinous process. In some embodiments, system 100 comprises interspinous attachment device 155 coupled to an existing crosslink.

In some embodiments, a crosslink 105 may have a first end 110 and a second end 115 in opposition to one another. A distance between first end 110 and second end 115 may generally correspond to a distance between two spinal fixation rods 120A and 120B fixed adjacent a spinal column of a patient. A first crosslink connector 125A and a second crosslink connector 125B may be provided to fixedly connect first end 110 and second end 115 of crosslink 105 to spinal rods 120A and 120B. First crosslink connector 125A and second crosslink connector 125B may have a housing 130 with a first end 135 and a second end 140 in opposition to one another. First end 135 may have a selectively attachable portion 145 to one of the spinal fixation rods 120A, 120B. Second end 140 may have a selectively attachable portion 150 to one of first end 110 and second end 115 of crosslink 105. Interspinous attachment device 155 may have a crosslink attachment portion 160 and a spinous process attachment portion 165. Crosslink attachment portion 160 may be configured to attach to crosslink 105 extending between two spinal fixation rods 120A, 120B. In one embodiment, this attachment may occur at a central or midline portion of crosslink 105 in a modular system. Mechanically cross-linking spinal fixation rods 120A, 120B improves torsional stability as well as helping prevent screw pull out. After spinal fixation rods 120A, 120B are placed, crosslink 105 may be placed from rod 120A to rod 120B and tightened.

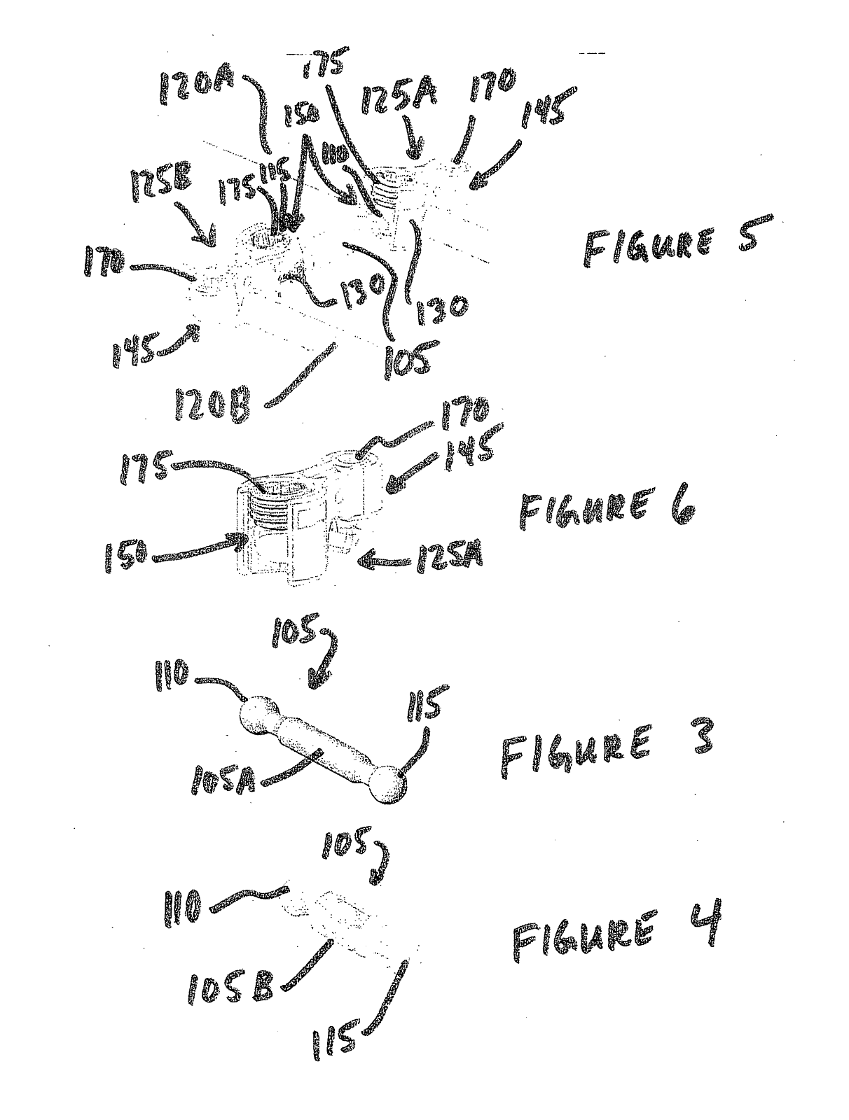

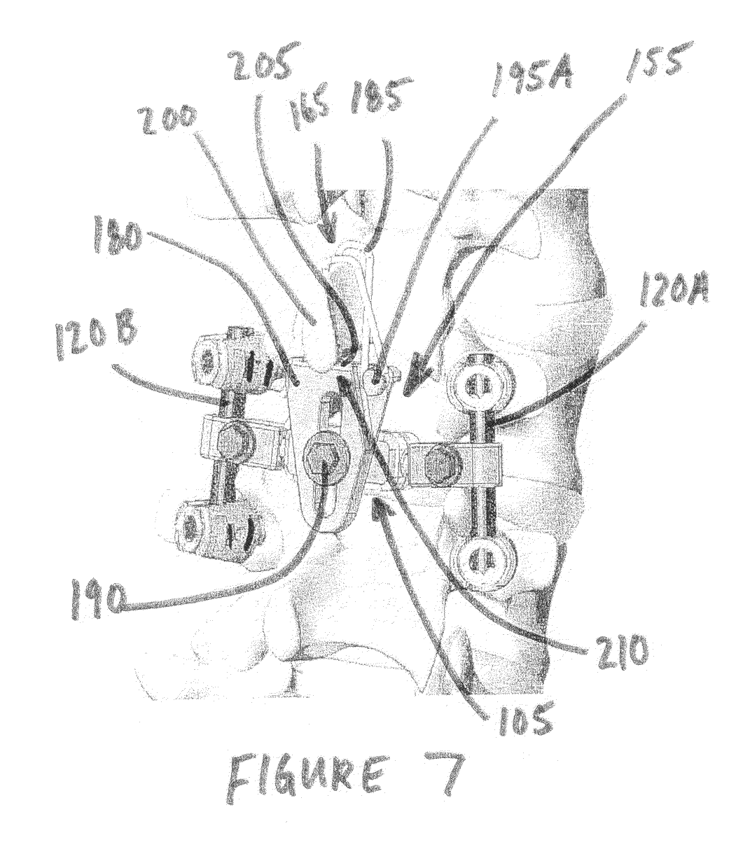

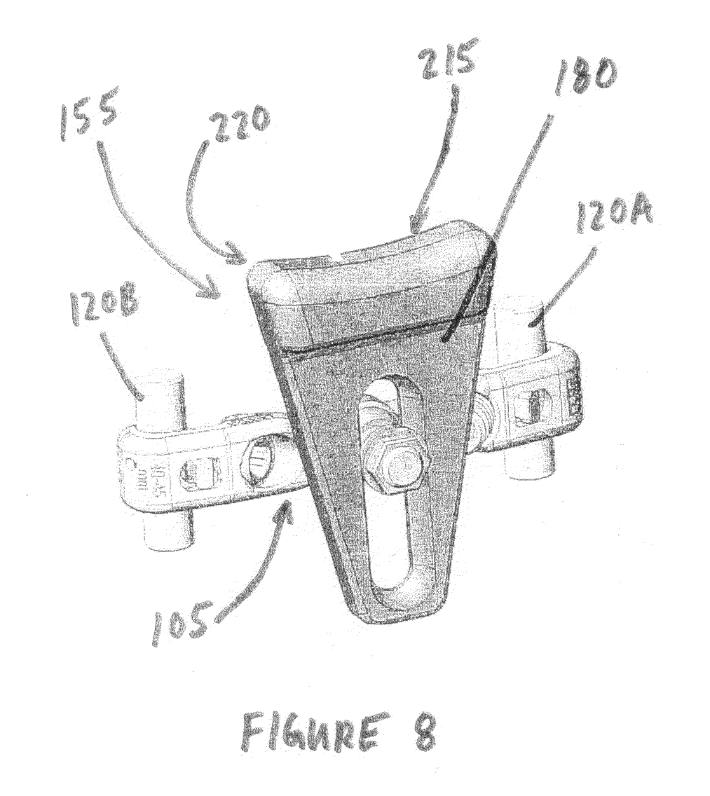

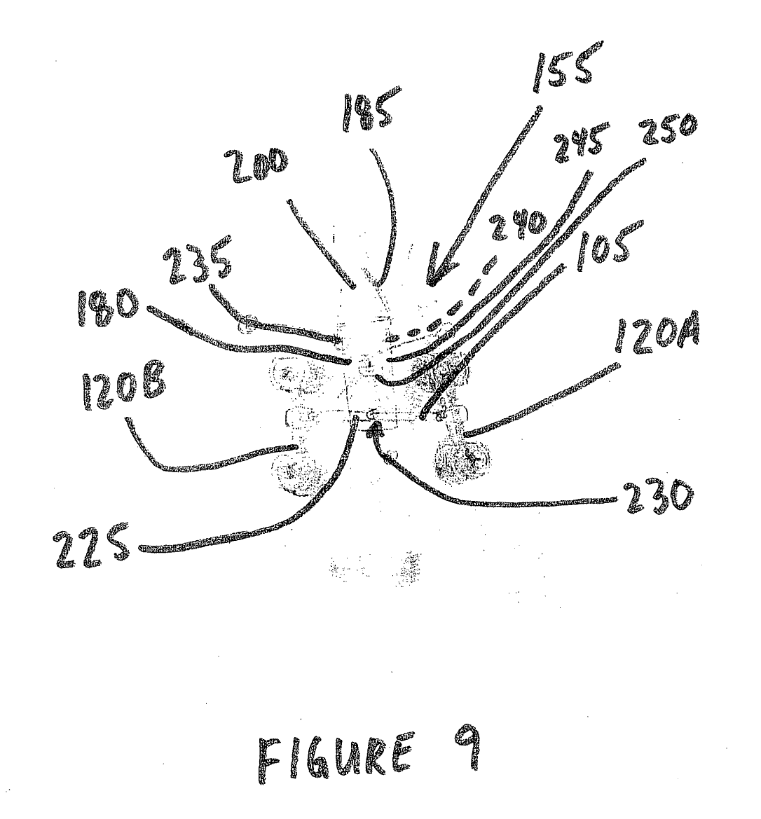

In one embodiment, crosslink 105 may have a fixed length 105A between first end 110 and second end 115 as illustrated, for example, in FIG. 3. Fixed length 105A may include a solid bar extending between first end 110 and second end 115. In another embodiment, crosslink 105 may have a selectively variable length 105B between first end 110 and second end 110 as illustrated, for example, in FIG. 4. Variable length 105A may include an adjustable bar extending between first end 110 and second end 115. In either embodiment, the length of crosslink 105 may be selected to substantially correspond with a width between two spinal fixation rods 120A, 120B together with the dimensions of crosslink connectors 125A, 1258 used to fixedly attach crosslink 105 to spinal fixation rods 120A, 120B.

First end 110 and second end 115 may include similar or different geometries to one another. In specific embodiments, the geometries of each of first end 110 and second end 115 may be a substantially spherical, ovoid, and/or ellipsoid end defining a protuberance extending from an extension created by fixed length 105A or variable length 1058. Spherical end 110, 115 have a roughened surface or coating, and in a particular embodiment may be grit blasted, for greater frictional engagement.

In an embodiment, and referring to FIGS. 5 and 6, each of crosslink connectors 125A, 125B may include a first fastener such as a set screw 170 engaging housing 130 at selectively attachable portion 145 so as to selectively retain one of the spinal fixation rods 120A, 120B. Set screw 170 may include an oversized cut to a bottom portion to better engage with first and second ends 110, 115 of crosslink 105. For example, set screw 170 may have a spherical or otherwise curved shape to the bottom portion thereof to engage with spherical or otherwise curved ends 110, 115. A second fastener such as a set screw 175 may threadably engage housing 130 at selectively attachable portion 150 so as to selectively retain either first end 110 or second end 115 of crosslink 105. As illustrated in FIG. 5, for example, crosslink 105 may be disposed in a generally perpendicular configuration to each of generally parallel spinal fixation rods 120A, 120B. In other embodiments, crosslink 105 may be disposed at various angles with respect to spinal fixation rods 120A, 120B due to the spherical connection between first end 110 or second end 115 and attachment portions 145, 150. Such an arrangement may be necessary, for example, to accommodate a patient's anatomy or other spinal implant constructs.

In one embodiment, interspinous attachment device 155 may include a block 180 (or anchor 180) and a ligament replacement band 185. Ligament replacement band 185 may be used to replace and/or to supplement a patient ligament. In some embodiments, block 180 includes a throughbore extending transversely through a central portion together with at least one fastener 190 so as to form crosslink attachment portion 160. In some embodiments, one or more anchor points 195A, 1958 may be disposed in block 180 toward a spinous process 200 when block 180 is attached to crosslink 105. In an embodiment, anchor points 195A, 195B together with ligament replacement band 185 may form spinous process attachment portion 165. Anchor points 195A, 1958 may include a hole and a fastener, such as a threaded hole and a set screw, to lock ligament replacement band 185 to block 180. Anchor points 195A, 195B may include pre-looped or pre-mounted connections with ligament replacement band 185. Anchor points 195A, 1958 may include crimpable portions sized for attachment with ligament replacement band 185.

Material selection of interspinous attachment device 155 has importance as a load sharing device and may involve consideration of potential wear debris from bone contact and failure of device 155. Prolonged loading and contact with bone may cause wear debris, failure of device 155, or a combination of both. In one embodiment, the modulus of elasticity of a material may be the same or substantially similar to natural bone. For example, PEEK is a good material for highly compressive loads. When the load is not mainly compressive, a titanium alloy may be a better choice than PEEK as the titanium alloy has good biocompatibility and withstands higher cantilevered loading conditions. Other materials may include stainless steel, implantable plastic, or other materials, with adequate strength.

Material selection for a ligament replacement, such as ligament replacement band 185, may involve consideration of the normal loading conditions of a functional spinal unit (FSU). The ligament replacement should withstand the normal loading conditions of the FSU. Consideration may be given to the mechanical properties of the ligament material. These properties may include, but are not limited to, tensile strength, creep, elasticity, resiliency, and crimp. High strength fibers may exhibit a desired strength for the ligament. However, high strength fibers may be too stiff and lead to damage in surrounding tissue. Less stiff polymers, including polyester (PET), and nylon, may exhibit good crimp morphology. A PET cable may be a good overall choice for biocompatibility, strength, and a strong positive clinical history. In some embodiments, a titanium or stainless steel cord or cable may be used.

In another embodiment, and referring to FIG. 7, an interspinous attachment device 155 may further include a spinous process support 205 formed by interspinous attachment device 155 for engagement with one portion of spinous process 200 with spinous process attachment portion 165 surrounding another portion of spinous process 200. In addition to providing support with ligament replacement band 185 during flexion, spinous process support 205 provides fixation to prevent movement of spinous process 200 once contact is made during extension. As illustrated, spinous process support 205 has a generally linear interface 210 with spinous process 200. In alternative embodiments, spinous process support 205 may be curved to contour to the shape of spinous process 200 or may form other geometries to engage with spinous process 200.

In another embodiment, and referring now to FIG. 8, interspinous attachment device 155 may further include a spinous process support 215 formed by interspinous attachment device 155 for engagement with one end or portion of spinous process 200 without an spinous process attachment portion. As illustrated, spinous process support 215 has a generally linear or concave interface 220 to engage spinous process 200. Spinous process support 205 acts as a stop to prevent movement of spinous process 200 once contact is made during extension. Spinous process support 215 may be curved to contour to the shape of spinous process 200 or may form other geometries to engage with spinous process 200. In the embodiment shown in FIG. 8, block 180 may include an elongate slot disposed therethrough for coupling interspinous attachment device 155 to crosslink 105. In this manner, the position of block 180 relative to crosslink 105 and thus relative to the patient anatomy can be altered. In some embodiments, the attachment mechanism for coupling block 180 to device 155 fixedly couples the two components in a desired orientation and position. In an alternative embodiment, block 180 has a different shaped and/or sized slot for coupling device 155 to crosslink 105.

In another exemplary embodiment, and referring to FIG. 9, interspinous attachment device 155 may further include a cross-hole, slot, or groove 225 extending across block 180 in a direction substantially perpendicular to the direction of engagement with spinous process 200. A fastener 230, such as a set screw 230, may be provided to lock block 180 to crosslink 105. Ligament replacement band 185 may be pre-attached to block 180 at a location 235, which may be disposed at or adjacent an end away from groove 225 and toward spinous process 200. Block 180 may include a cross-hole 240 extending though a middle portion 245. Band 185 may be positioned to extend around spinous process 200 and be threaded through cross-hole 240. Another fastener 250, shown in FIG. 9 as a set screw 250, may be positioned adjacent cross-hole 240 to selectively lock-down band 185. Once band 185 is secured to block 180, any excess length extending away from set screw 250 may be trimmed off or otherwise removed.

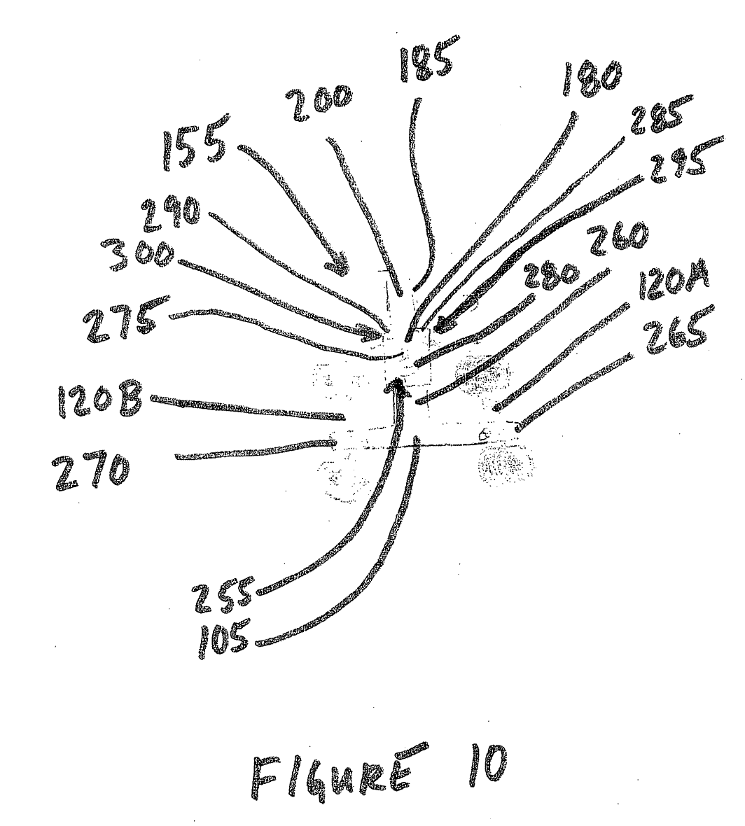

In an embodiment, and referring to FIG. 10, interspinous attachment device 155 may include crosslink 105 having a T-shape with a post 255 at one arm 260 as well as first end 110 and second end 115 at the other two arms 265, 270, respectively. Block 180 may include a slot 275 corresponding to post 255. Slot may have a length to allow post 255 to slide as block 180 is repositioned away from or toward spinous process 200. A fastener 280, such as a locking screw 280 may be positioned to operatively engage with post 255 and lock-down block 180 in a desired position relative to spinous process 200. Band 185 may pass through holes 285, 290 and crimps 295, 300 may be provided to maintain tension of band 185 extending around spinous process 200 and in engagement with crimps 295, 300.

In one embodiment, and referring to FIG. 11, interspinous attachment device 155 may include crosslink 105 having a double-L shape with ends 305 disposed toward spinous process 200 forming a clamp 310 as interspinous attachment device 155. Ends 315 disposed away from ends 305 may provide first end 110 and second end 115, which are attachable to spinal fixation rods 120A, 120B.

As shown in phantom at 320, double-L shape with ends 305 may be a single unit with a double or bifurcated y-shaped post. This configuration may increase torsional stability and may interfere with spinous process 325 if not removed at the screw-instrumented level. A single band 185 may limit flexion and extension if multiple crimps 330 are used through cross-holes 335 at ends 305 adjacent clamp 310.

In an embodiment, and referring to FIG. 12, interspinous attachment device 155 may include rods 340 extending from pedicle screws 345. Through-holes 350 and corresponding crimps 355 may be provided to attach bands or cables 185 extending between rods 340. This configuration may be used as alternate attachment, such as when a screw is not desired or possible, or to share a load with a screw. In one embodiment, bands 185 may extend from rods 340 to wrap around transverse processes. This could be provided in a sublaminar configuration. Bands 185 may further extend around spinous process 200.

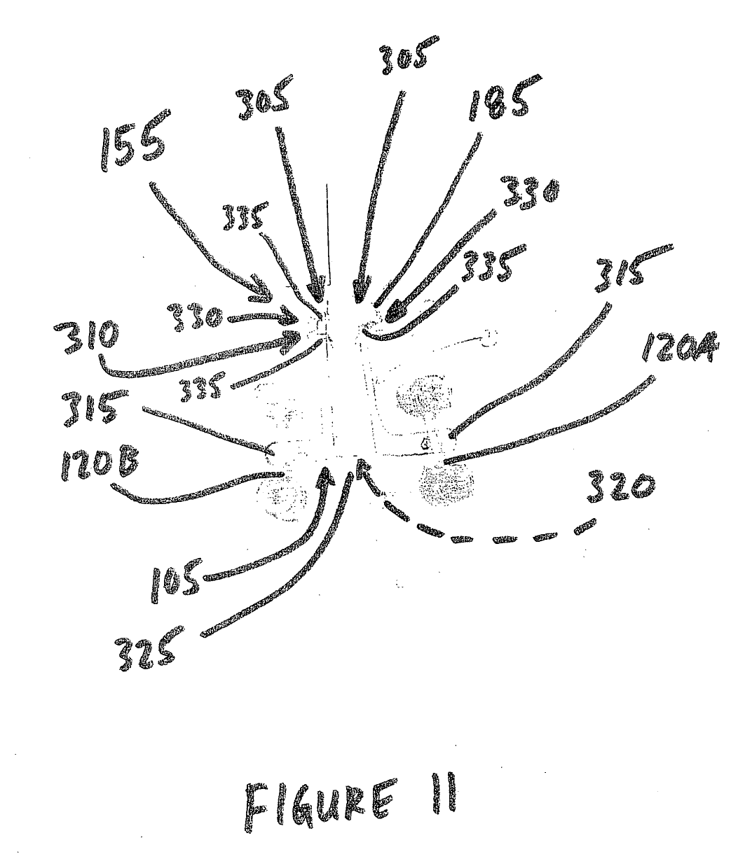

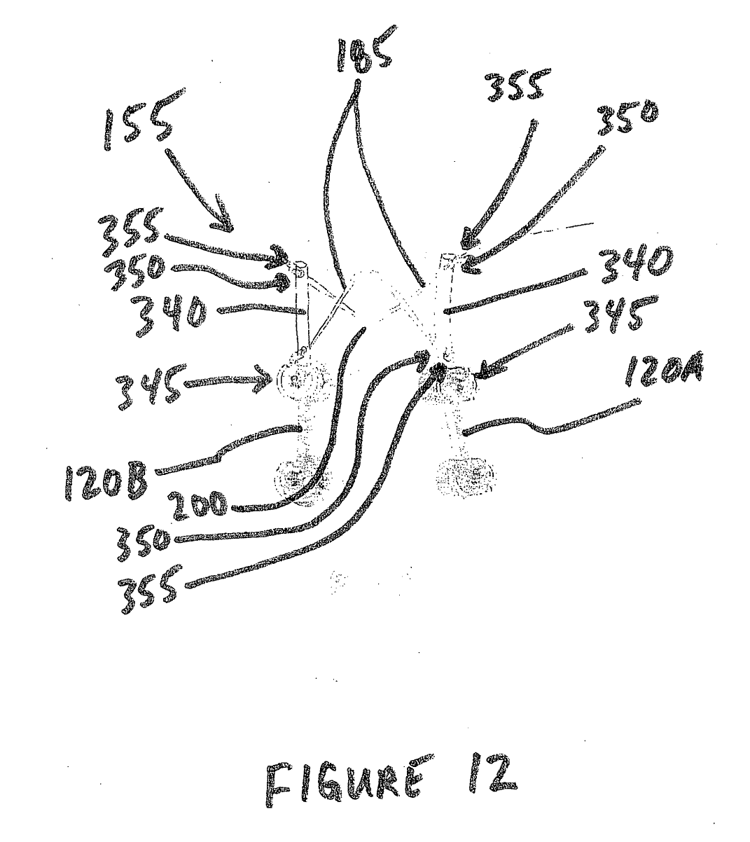

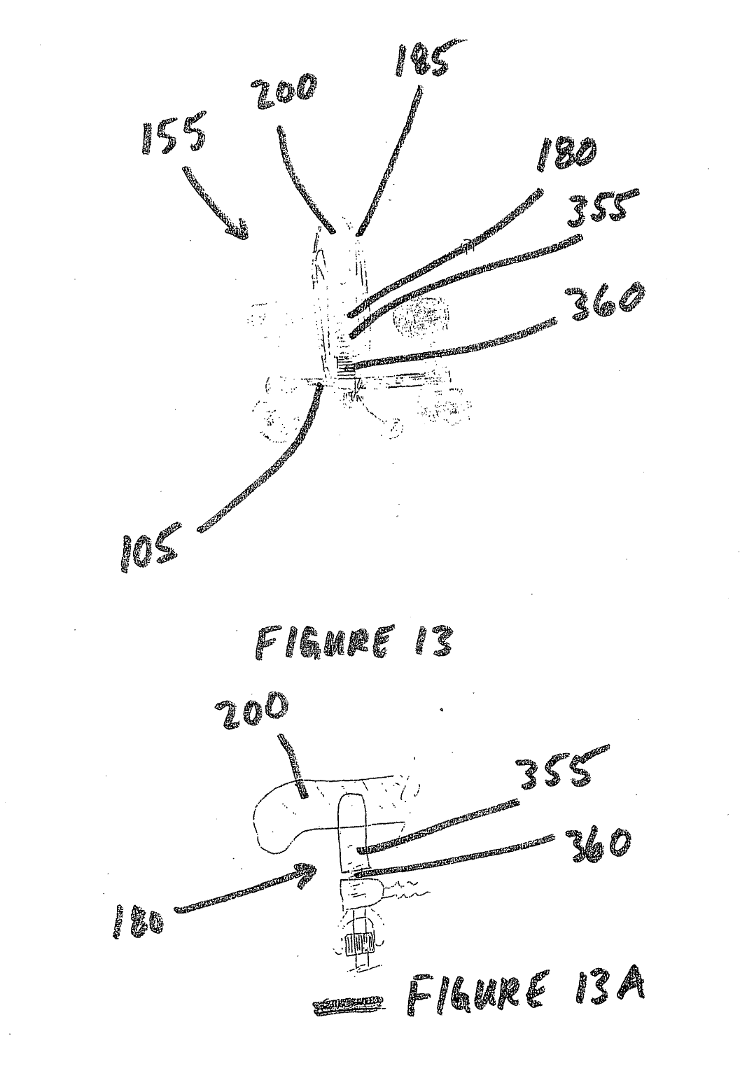

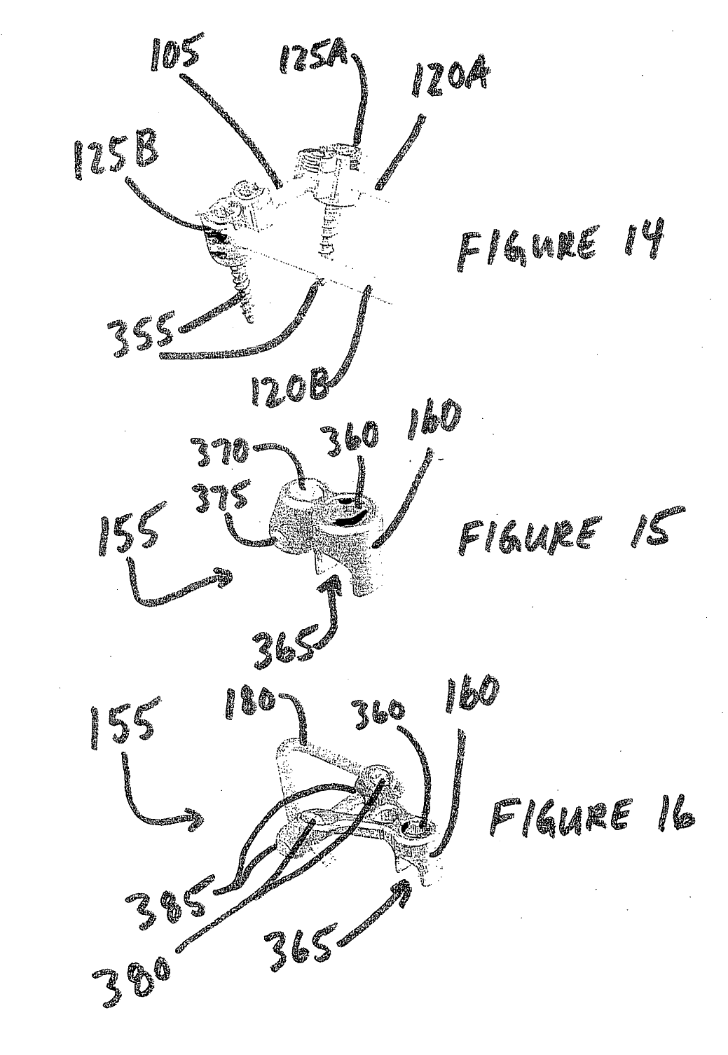

In an embodiment, and referring to FIGS. 13 and 13A, interspinous attachment device 155 may include a multi-piece block 180. In the embodiment shown, block 180 is a two piece block having a female threaded portion 355 and a male threaded portion 360, which are selectively engagable with one another to selelctively adjust the height of block 180. In one embodiment, the height of block 180 may be adjusted prior to attachment to crosslink 105 or placement against spinous process 200. Band 185 may be provided as a separate item, not pre-attached to crosslink 105 or block 180. Band 185 may be looped around spinous process 200, block 180, and crosslink 105, and may be crimped in one location to selectively attach interspinous attachment device 155 to spinous process 200. Referring now to FIG. 14, in this embodiment pedicle screws 355 extend directly from first crosslink connector 125A and second crosslink connector 125B. In this configuration, first crosslink connector 125A and second crosslink connector 125B attach directly to crosslink 105, pedicle screws 335, and spinal fixation rods 120A and 120B. This provides a compact design and does not require additional anchor hardware so as to reduce screw placement and trauma by anchoring spinal fixation rods 120A and 120B as well as crosslink 105 with the same pedicle screws 335.

In exemplary embodiments, and referring to FIGS. 15 and 16, various types of interspinous attachment devices 155 may be provided with crosslink attachment portion 160 and spinous process attachment portion 165. In FIG. 15, a set screw 360 threadably engages crosslink attachment portion 160 and selectively attaches interspinous attachment device 155 to a crosslink (not shown) within passageway 365. A set screw 370 threadably engages spinous process attachment portion 165 and selectively secures a band (not shown) within passageway 375. In FIG. 16, set screw 360 threadably engages crosslink attachment portion 160 and selectively attaches interspinous attachment device 155 to a crosslink (not shown) within passageway 365. One or more set screws 380 may threadably engage spinous process attachment portion 165 and selectively secures band 180 within passageways 385. While the depicted embodiments have been described using set screws 360, 380, alternative fasteners may be used within the scope of the present disclosure including, for example, pins, stakes, collets or other snap-in connectors, or the like.

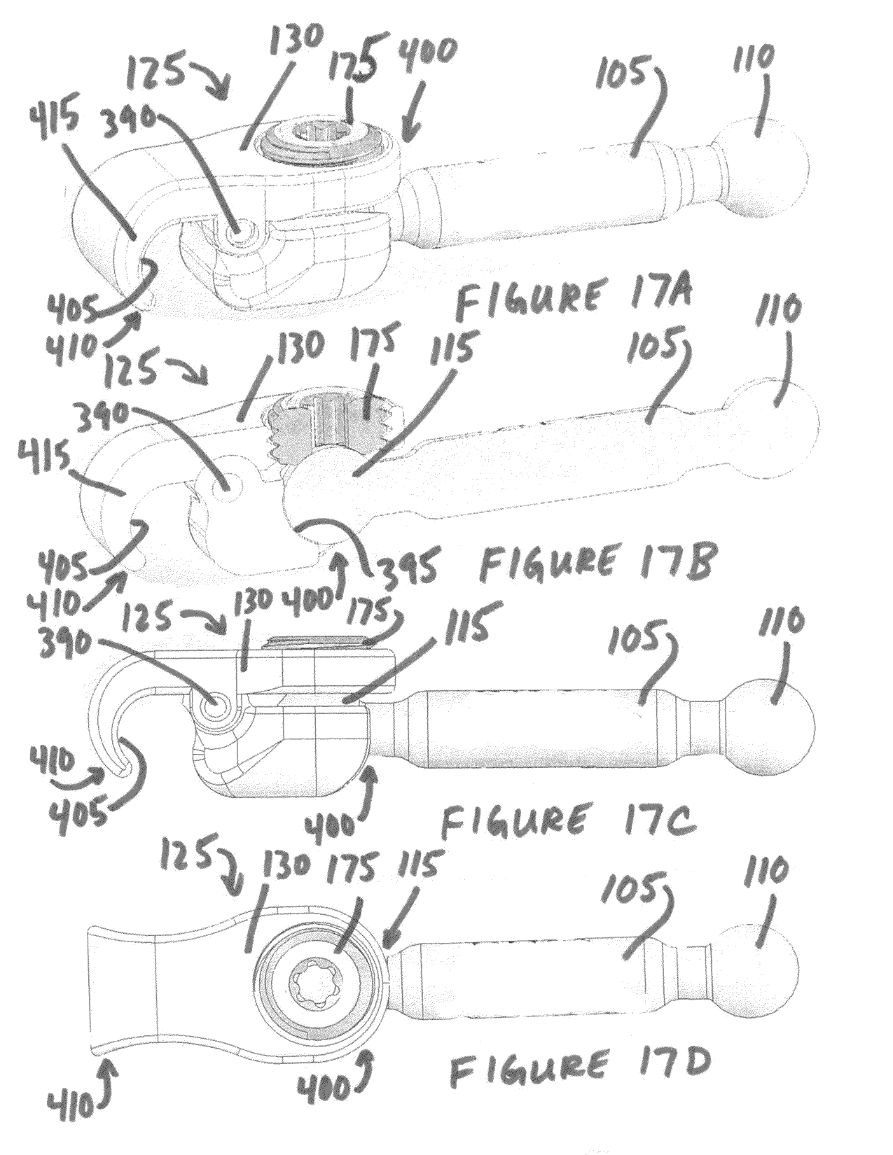

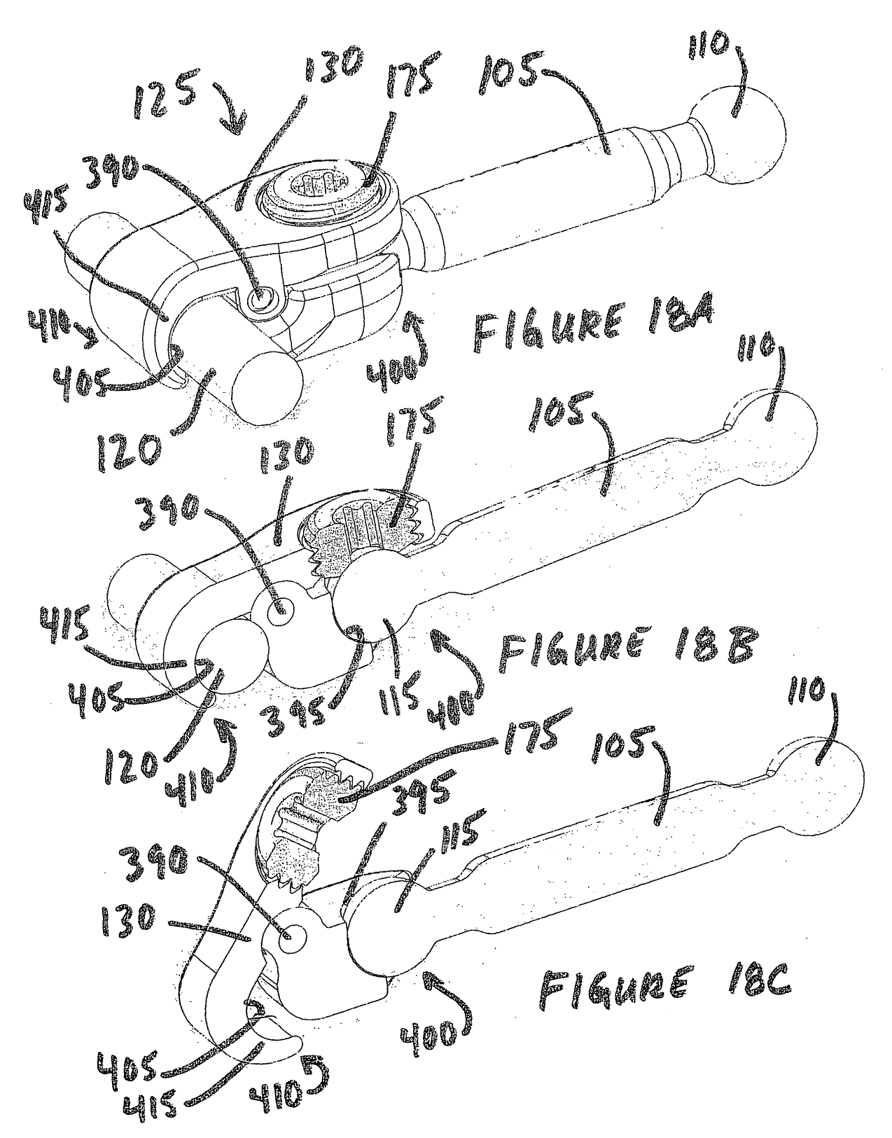

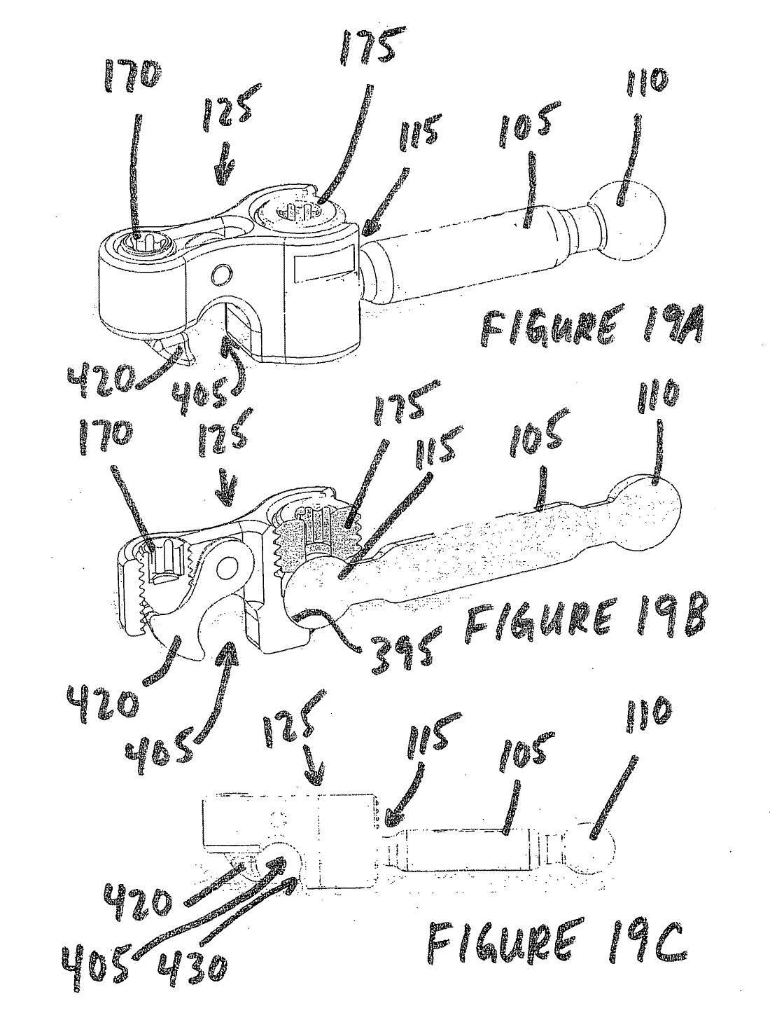

In an embodiment, and referring to FIGS. 17A-17D and 18A-18C, crosslink connector 125 (which may be implemented as either first crosslink connector 125A or second crosslink connector 125B) includes a pivot 390 to provide a compact clamping mechanism to selectively fix crosslink 105 and one of spinal fixation rods 120A, 120B to one another. This configuration may provide a simple device and small profile.

A pocket 395 formed in housing 130, with a shape and dimensions to selectively retain one of ends 110, 115 of crosslink 105, may be provided at one end 400 of crosslink connector 125. In embodiments with spherical ends 110, 115, pocket 395 may be spherical. Pocket 395 may be grit blasted or have other texturing characteristics for greater frictional engagement. A rod pocket 405 with dimensions to selectively retain one of spinal fixation rods 120A, 120B may be provided elsewhere in connector 125, such as at or near an opposing end 410 of crosslink connector 125. Rod pocket 405 may be formed by a pivot arm 415 selectively rotatable with respect to housing 130 with pocket 395. Pivot arm 415 may be actuated to allow placement of one of ends 110, 115 into pocket 395. One of spinal fixation rods 120A, 120B may be placed into rod pocket 405. In this embodiment, a single screw 175 may be used to lock both sides of the clamping mechanism. Set screw 175 may be rotated to engage one of ends 110, 115 and, in turn, drive pivot arm 415 around pivot 390 to tightly clamp one of rods 125A, 125B. Threaded engagement of the set screw provides adequate force to prevent pullout of crosslink 105. In this configuration, one of ends 110, 115 is placed within pocket 395 prior to placement of one of rods 125A, 125B into rod pocket 405.

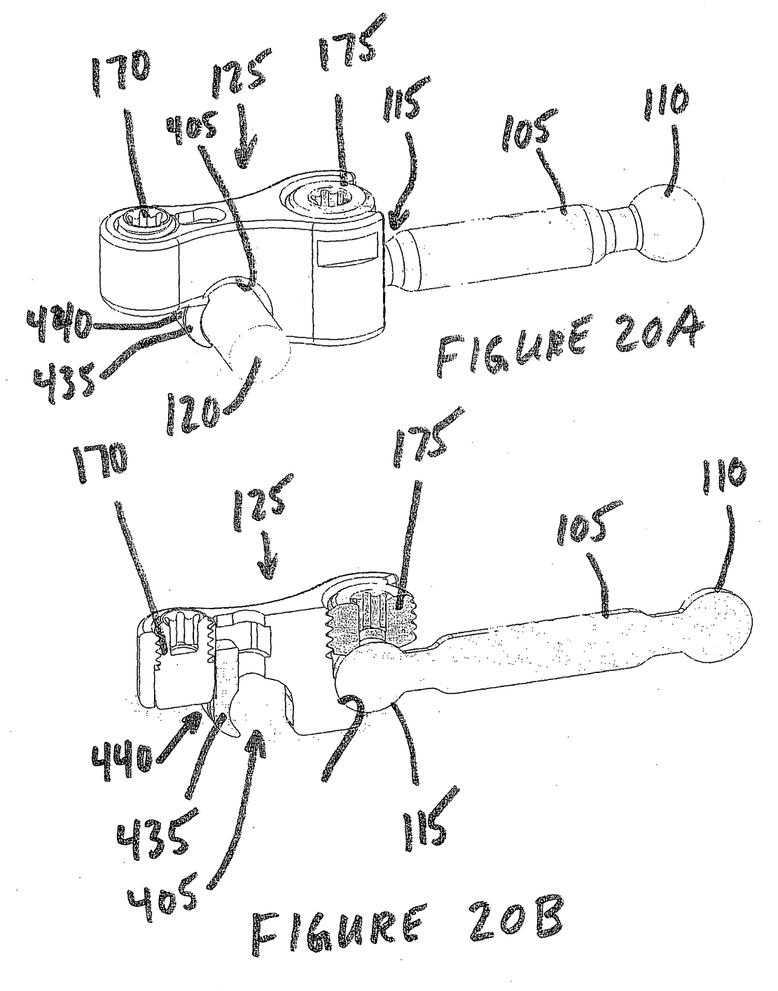

In another embodiment, and referring now to FIGS. 19A-19C, a swing arm 420 may engage with set screw 170 and selectively retain one of spinal fixation rods (not shown.) A swing arm pivot 425 may be provided in swing arm 420 to allow rotation of swing arm 420 into rod pocket 405 as set screw 170 is tightened. A dimple may be provided for small set screw to slip into when tightening set screw 170. Each of pocket 395 and rod pocket 405 separately retain crosslink 105 and one of the spinal fixation rods with separate ones of set screw 175 and set screw 170, respectively. Referring to FIG. 19C, there may be provided a bump or protrusion 430 to assist with swing arm 420 in retaining a spinal fixation rod.

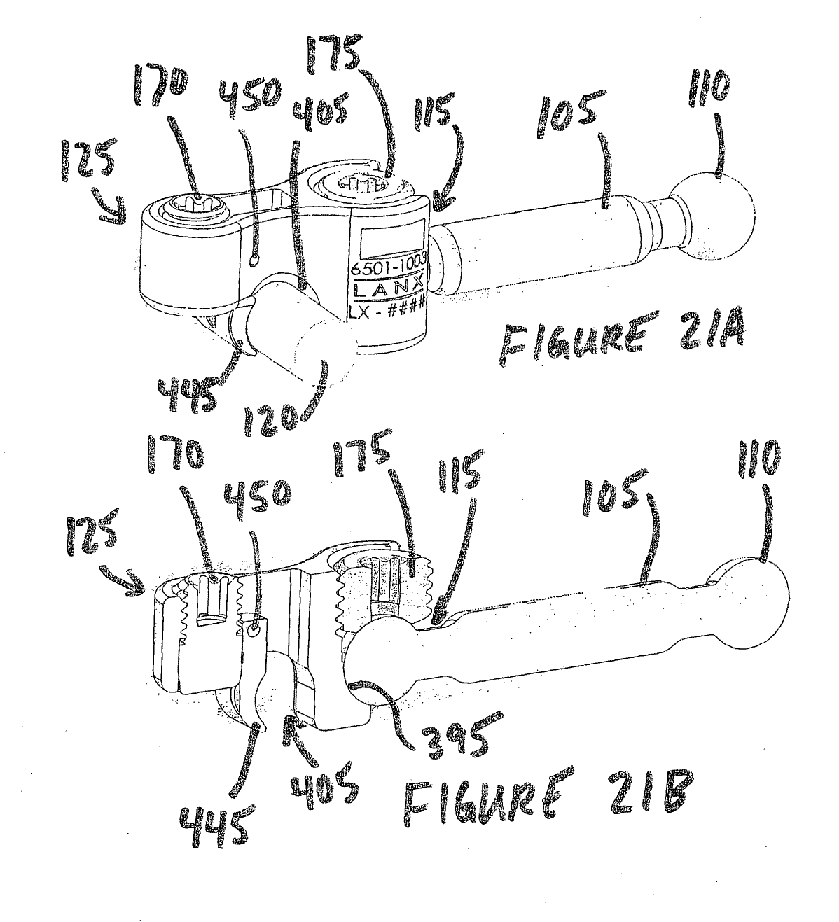

In one embodiment, and referring now to FIGS. 20A and 20B, a swing arm 435 may engage with set screw 170 and selectively retain one of spinal fixation rods (not shown.) A protrusion 440 on one side of swing arm 435 opposite to a side disposed toward rod pocket 405 may be provided on swing arm 435 to force swing arm 435 into rod pocket 405 when set screw 170 is tightened. Each of pocket 395 and rod pocket 405 separately retain crosslink 105 and one of the spinal fixation rods with separate ones of set screw 175 and set screw 170, respectively. Set screw 170 may include a buttress thread for engagement with swing arm 435. Swing arm 435 holds rod in compression. In an alternative embodiment, swing arm 435 may translate instead of rotate when set screw 170 is tightened into a threaded receiving hole in crosslink connector 125. The translation of arm 435 forces arm 435 towards rod 120 to engage rod 120 within rod pocket 405.

In one embodiment, and referring now to FIGS. 21A and 21B, a swing arm 445 extends between rod pocket 405 and set screw 170. A swing arm pivot 450 may be provided in swing arm 445 to allow rotation of swing arm 445 into rod pocket 405 as set screw 175 is tightened. In addition, a protrusion 455 on one side of swing arm 445 opposite to a side disposed toward rod pocket 405 may be provided on swing arm 435 to also force swing arm 435 into rod pocket 405 when set screw 175 is tightened. Each of pocket 395 and rod pocket 405 separately retain crosslink 105 and one of the spinal fixation rods with separate ones of set screw 170 and set screw 175, respectively. Set screw 170 may include a buttress thread for engagement with swing arm 445. Pivot 450 generally does not receive a great amount of clamping force as forces are primarily transferred through the swing arm 435 directly to a rod.

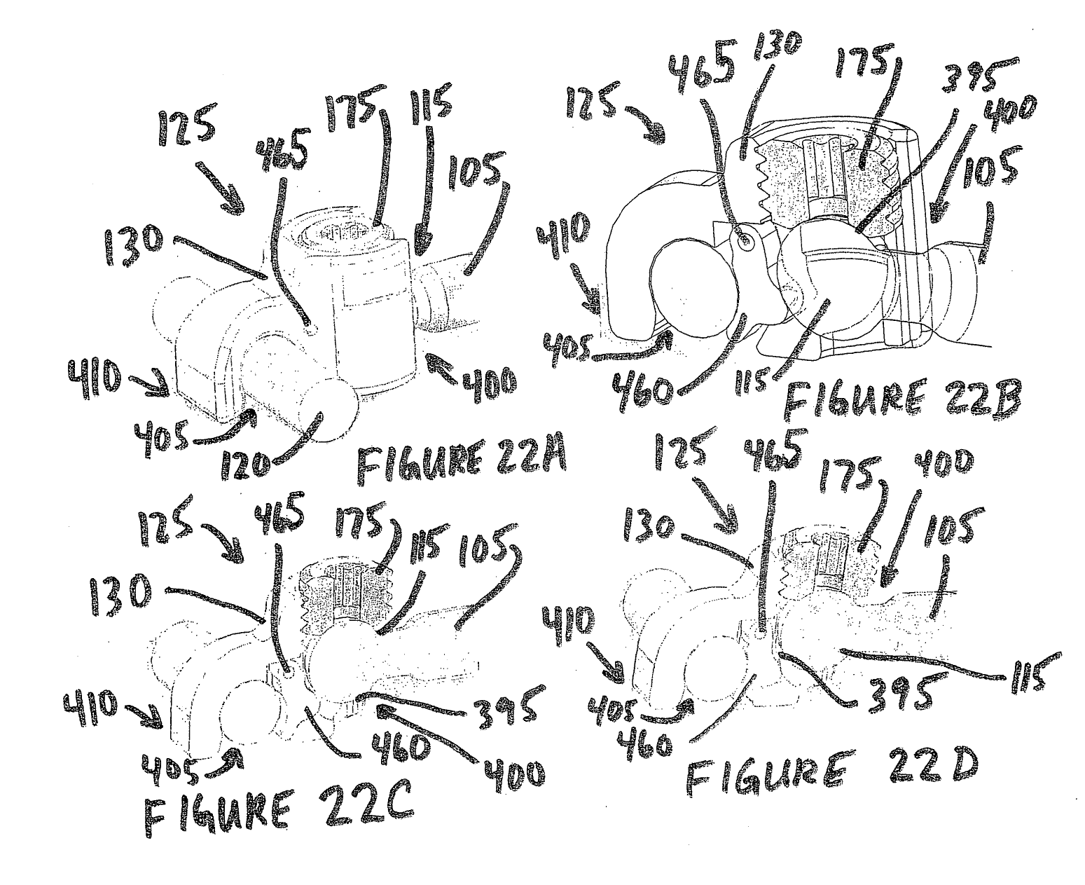

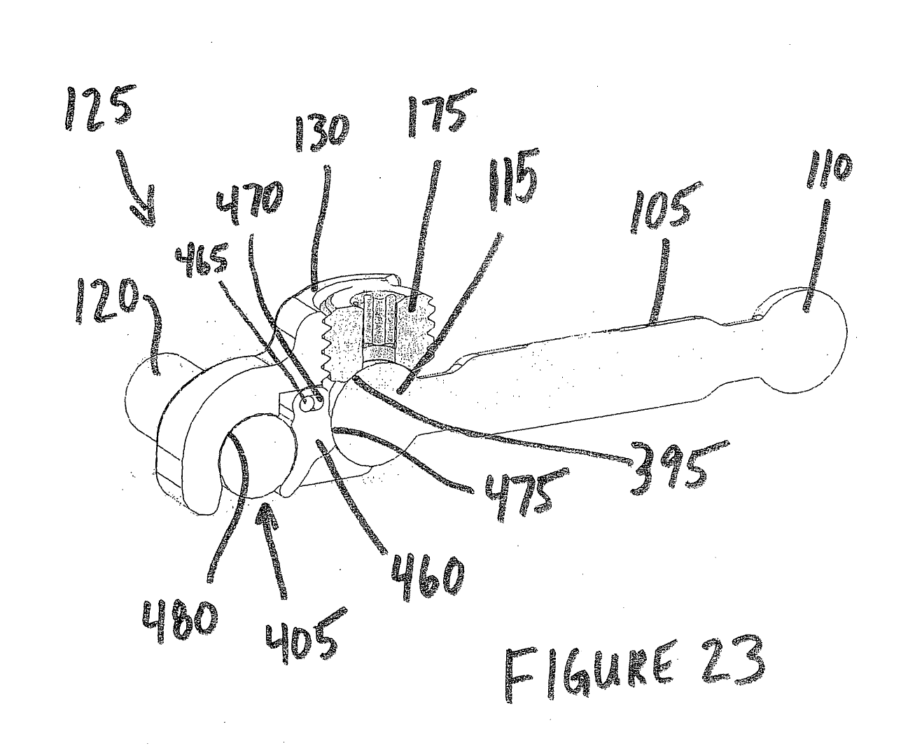

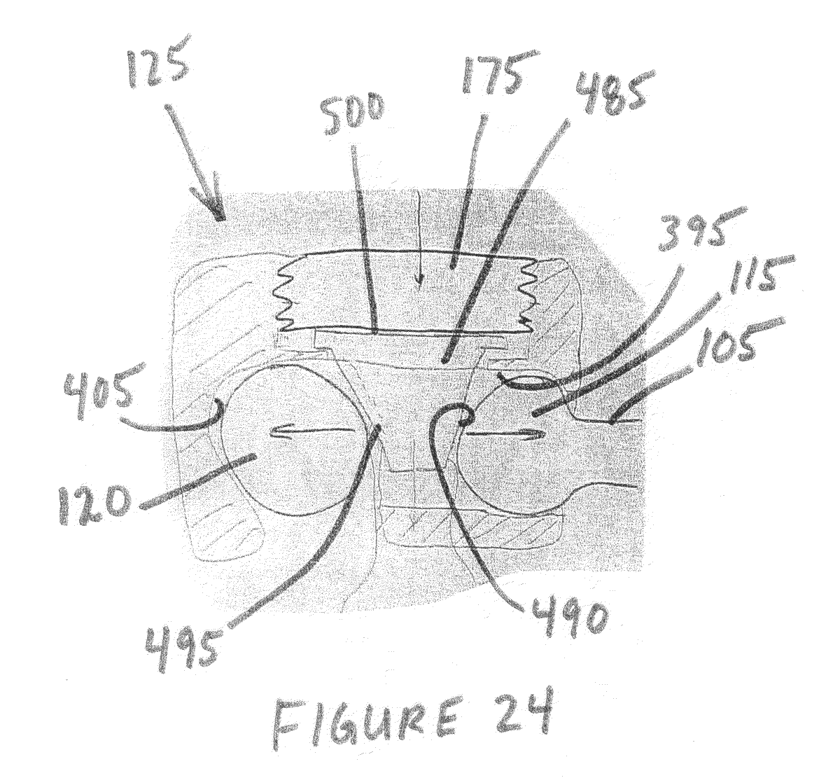

In another embodiment, and referring to FIGS. 22A-22D, crosslink connector 125 (which may be implemented as either first crosslink connector 125A or second crosslink connector 125B) includes a swing arm 460 with a pivot 465 disposed within housing 130 between spherical pocket 395 and rod pocket 405. Swing arm 460 may be configured to engage one of ends 110, 115 of crosslink 105 as well as one of rods 120A, 120B to provide a compact clamping mechanism to selectively fix crosslink 105 and one of spinal fixation rods 120A, 120B to one another. Pocket 395 may be formed in housing 130 with a shape and dimensions to selectively retain one of ends 110, 115 of crosslink 105 at one end 400 of crosslink connector 125. Rod pocket 405 with a shape and dimensions to selectively retain one of spinal fixation rods 120A, 120B may be provided at another location, such as at an end 410 of crosslink connector 125 in opposition to end 400. After placement of one of ends 110, 115 within pocket 395 and one of rods 125A, 1258 into rod pocket 405, the single set screw 175 may be actuated to drive one of ends 110, 115 into swing arm 460 and, in turn, drive swing arm 460 away from one of ends 110, 115 and toward one of rods 125A, 125B. Swing arm 460, ends 110, 115, and rods 125A, 125B may be sized such that tightening set screw 175 with adequate torque locks together one of ends 110, 115 and one of rods 125A, 125B in housing 130.

In another embodiment, and referring to FIG. 23, a slot 470 may be provided in swing arm 460 to surround pivot 465. Slot 470 allows greater movement and adjustment of swing arm 460. In this embodiment, pivot 465 may translate within slot 470 to allow translation of swing arm 460 in addition to rotation movement about pivot 465. In addition, swing arm 460 with slot 470 may include an increased surface area 475 to contact one of ends 110, 115 than a rotatable swing arm 445 with only generally rotatable translation rather than lateral translation. Swing arm 460 with slot 470 may also include an increased surface area 480 to contact one of rods 120A, 120B than a rotatable swing arm 445 with only generally rotatable translation rather than lateral translation. This increased surface area 475 and increased surface area 480 provides an increased clamping force between one of ends 110, 115 and one of rods 120A, 120B.

In one embodiment, and referring to FIG. 24, crosslink connector 125 (which may be implemented as either first crosslink connector 125A or second crosslink connector 125B) includes a wedge 485 disposed between pocket 395 and rod pocket 405. A crosslink inclined face 490 positioned toward pocket 395 may taper or have a curved, cupped face corresponding with one of ends 110, 115. A rod inclined face 495 positioned toward rod pocket 405 may taper or have a curved, cupped face corresponding with one of rods 120A, 120B. A set screw engagement face 500 is disposed toward set screw 175. Crosslink inclined face 490 and rod inclined face 495 are sized with respect to one of ends 110, 115 and one of rods 120A, 120B, respectively, to fixedly attach both to housing 130 after adequate torque is applied to set screw 170.

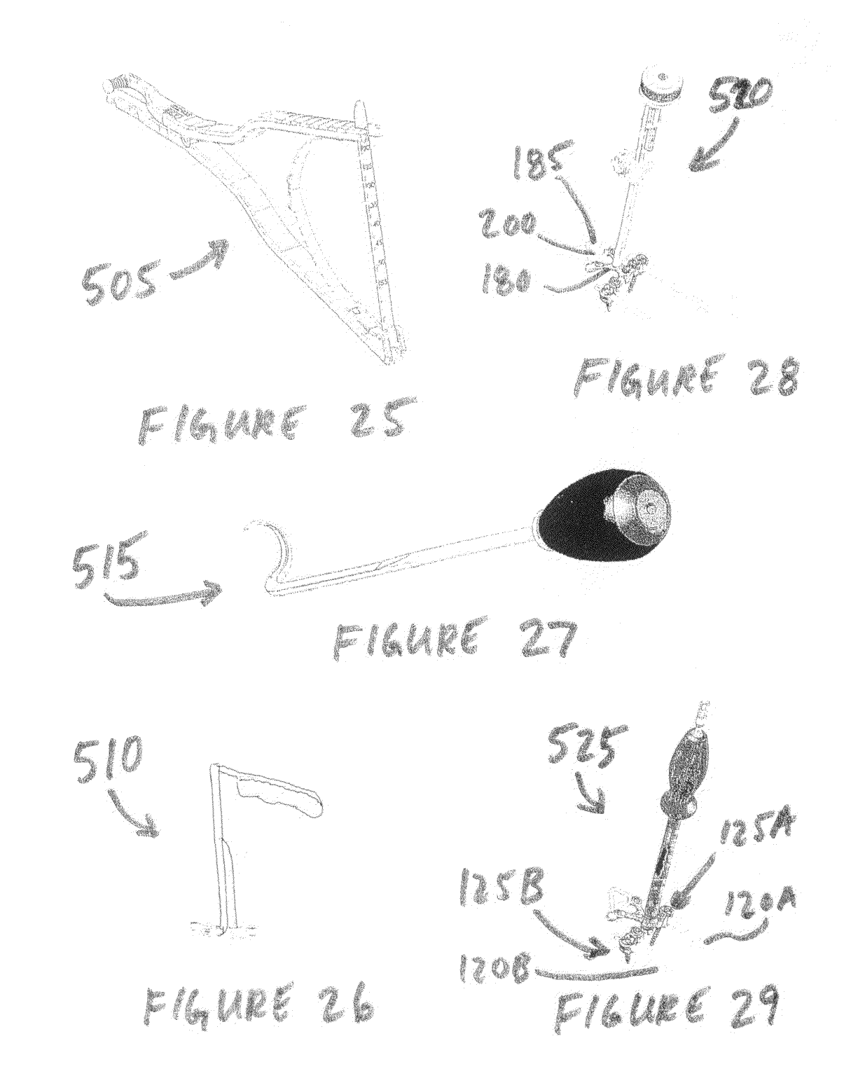

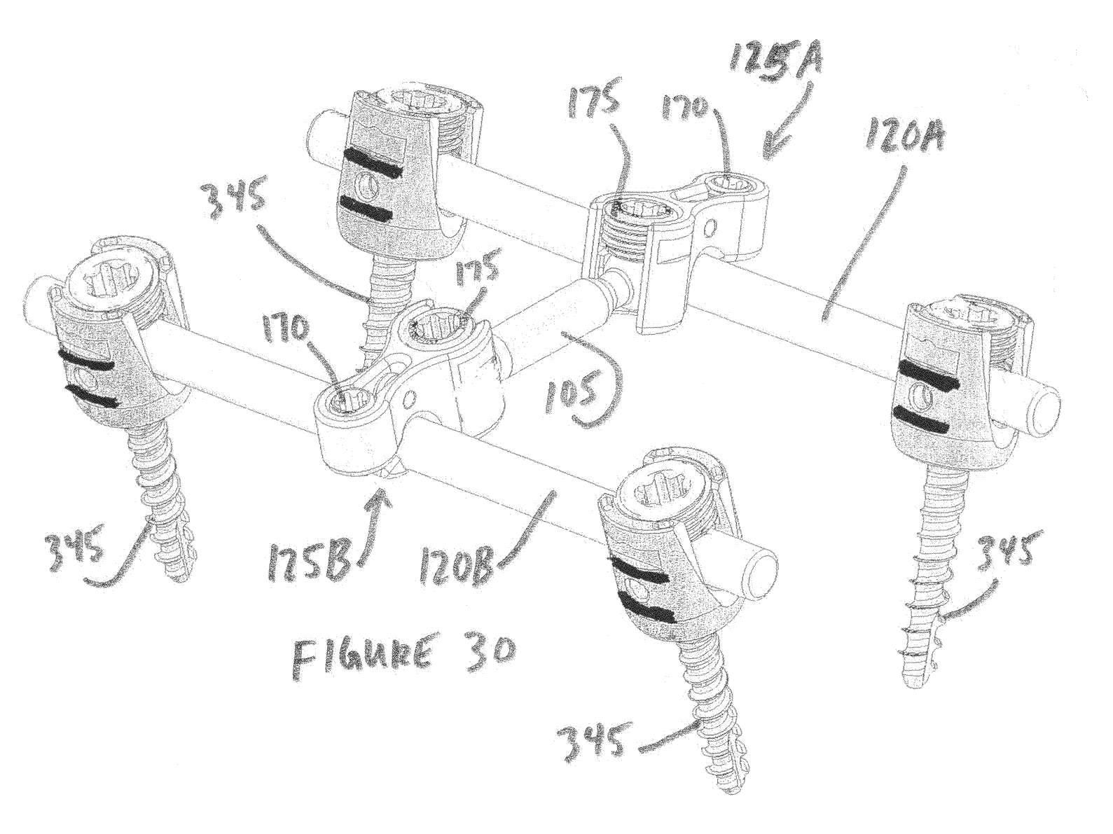

Referring now to FIG. 25, an implant calibrator 505 may be provided to select a correctly sized band, to correctly measure a crosslink length, to measure an interspinous attachment device length, and/or to provide other measurements A crosslink calibrator 510 (FIG. 26) may be provided to select a correctly sized crosslink for placement between a pair of rods. A cable pass-through device 515 (FIG. 27) may be provided to position band around a spinous process and relative to an interspinous attachment device. A cable tensioner 520 (FIG. 28) may be provided to impart a desired amount of tension on band 185 positioned around spinous process 200 and attached to block 180 of interspinous attachment device 155. A clamp inserter 525 (FIG. 29) may be provided to position crosslink connectors 125A, 125B with respect to spinal fixation rods 120A and 120B.

Referring now to FIG. 30, there is shown a crosslink 105 in attachment to spinal fixation rods 120A, 120B with crosslink connectors 125A, 125B, respectively. Pedicle screws 345 or other fixation devices may be positioned to support spinal fixation rods 120A, 120B. However, crosslink 105 does not require any additional fixation screws.

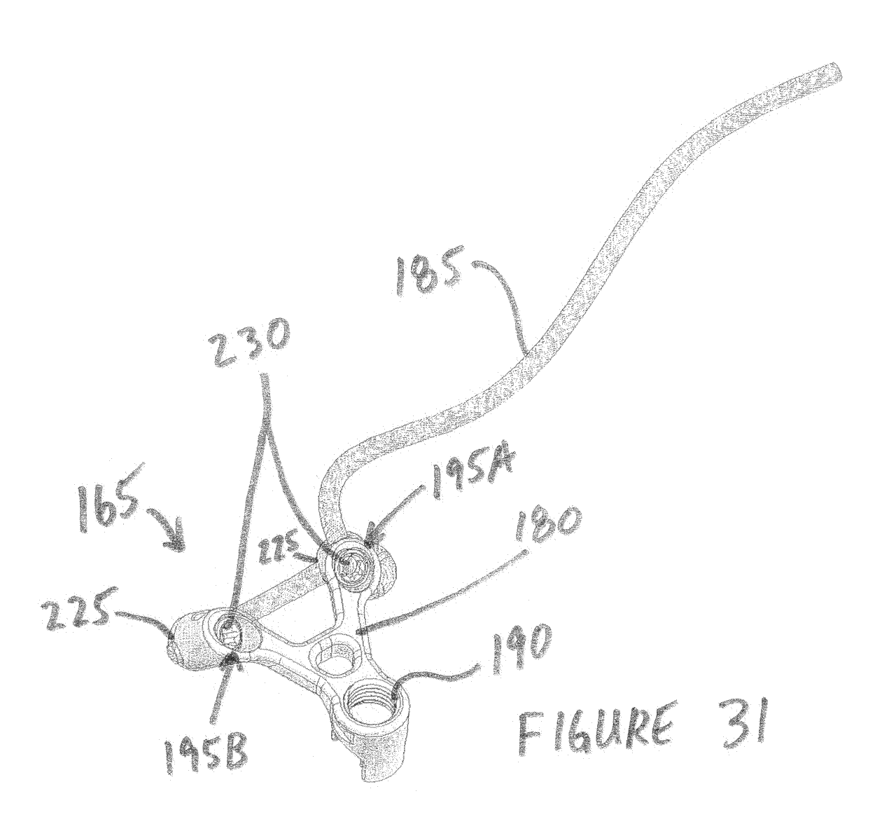

In FIG. 31 there is illustrated spinous process attachment portion 165 with block or anchor 180 having set screws 230 to retain band 185 in respective cross-holes 225. Threaded hole 190 is adapted to receive a threaded fastener to couple attachment device 165 to a crosslink.

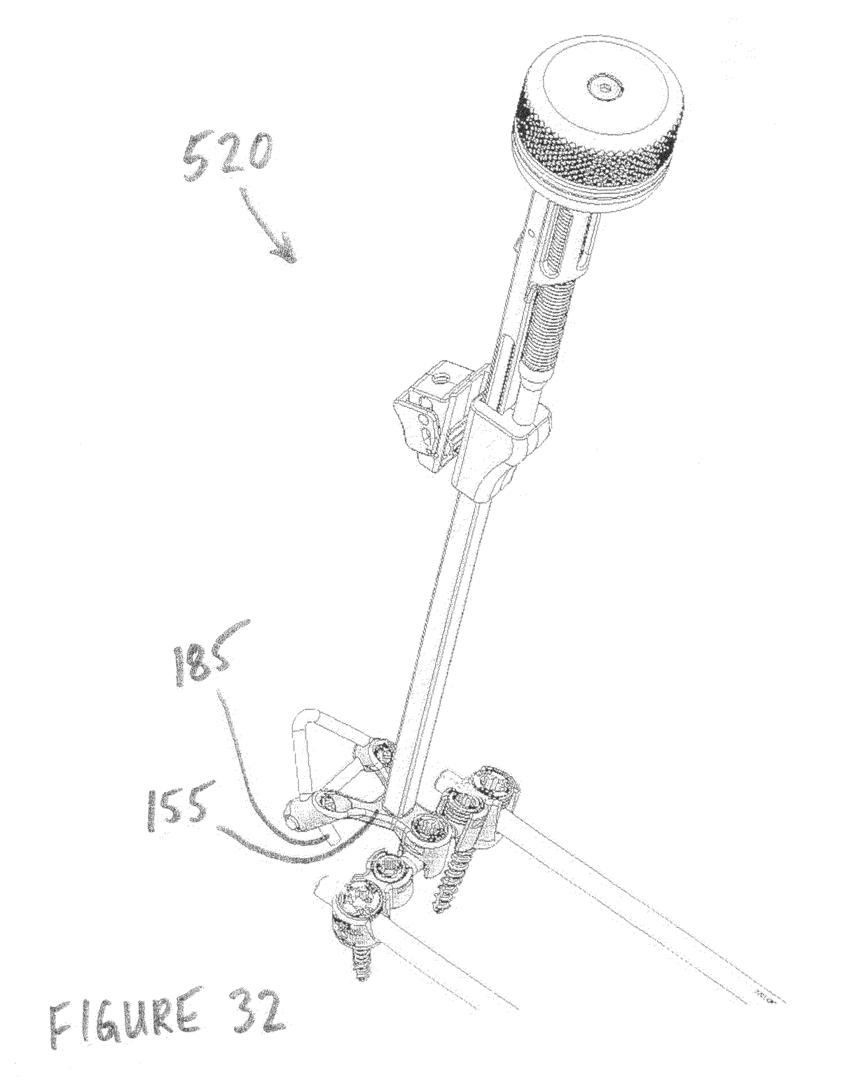

Similar to FIG. 28 above, there is shown cable tensioner 520 (FIG. 32) in engagement with interspinous attachment device 155 to attach with a free end of band 185 and apply a desired amount of tension. Once a desired amount of tension has been provided, tensioner 520 maintains the tension as band 185 is coupled to interspinous attachment device 155.

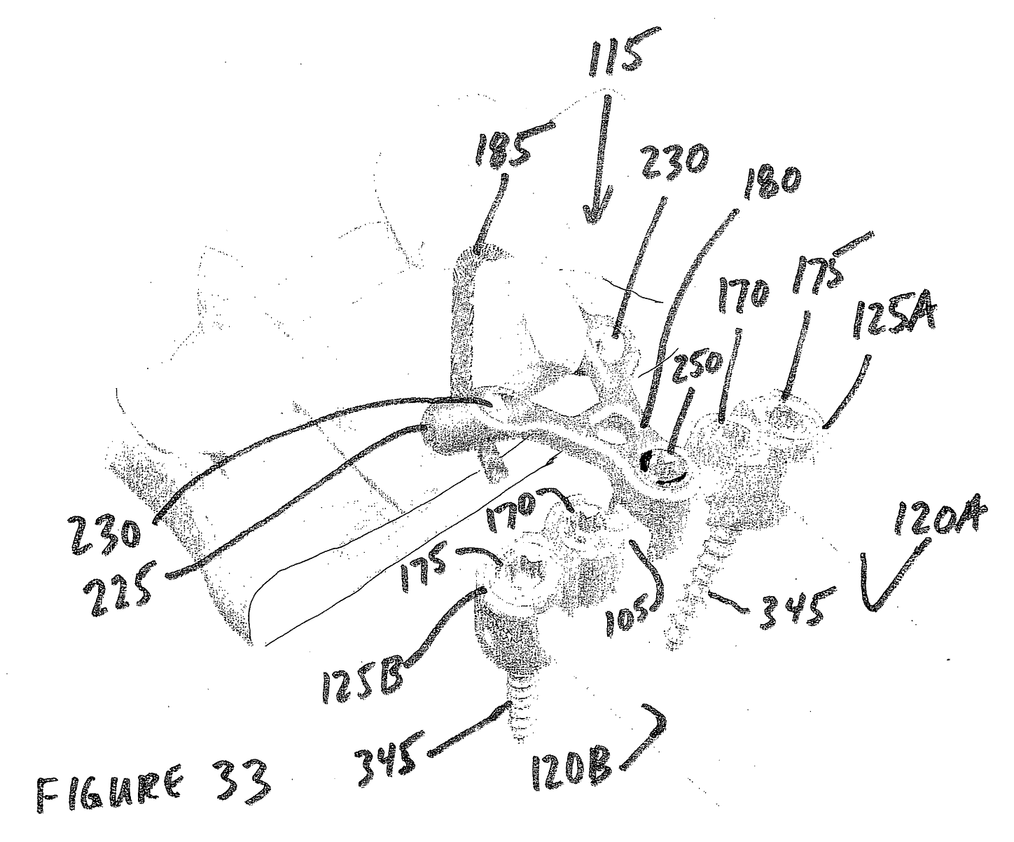

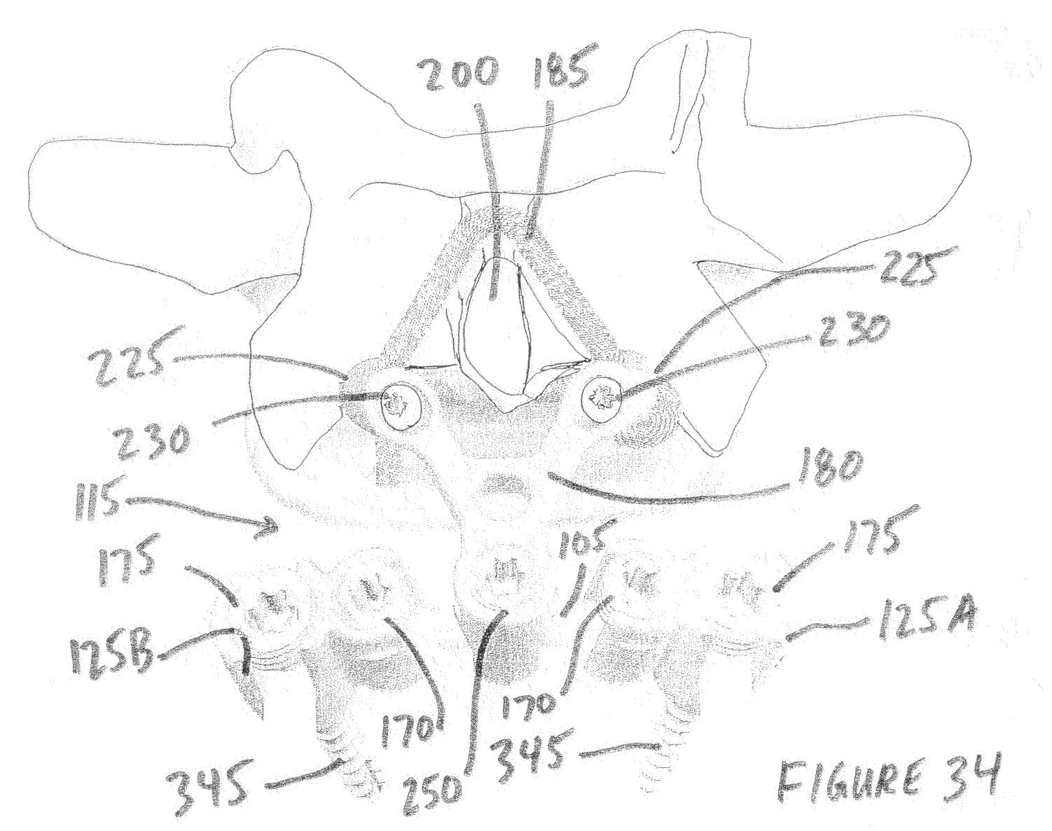

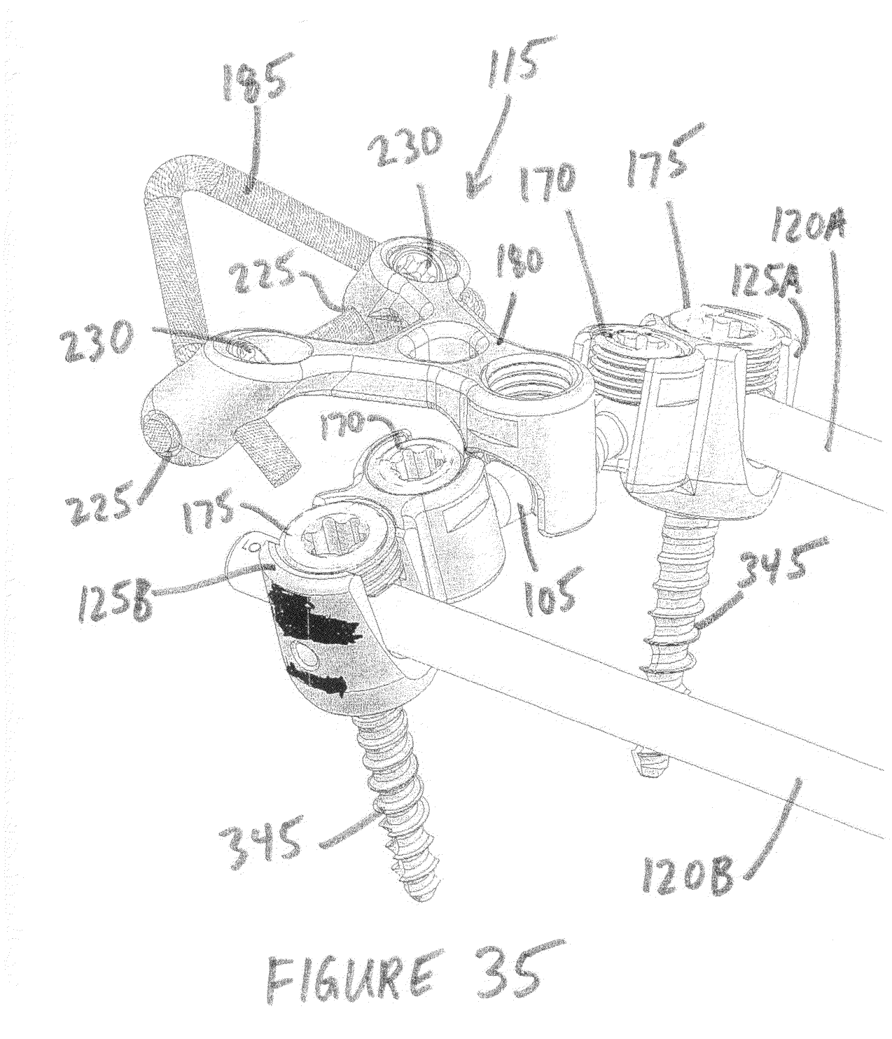

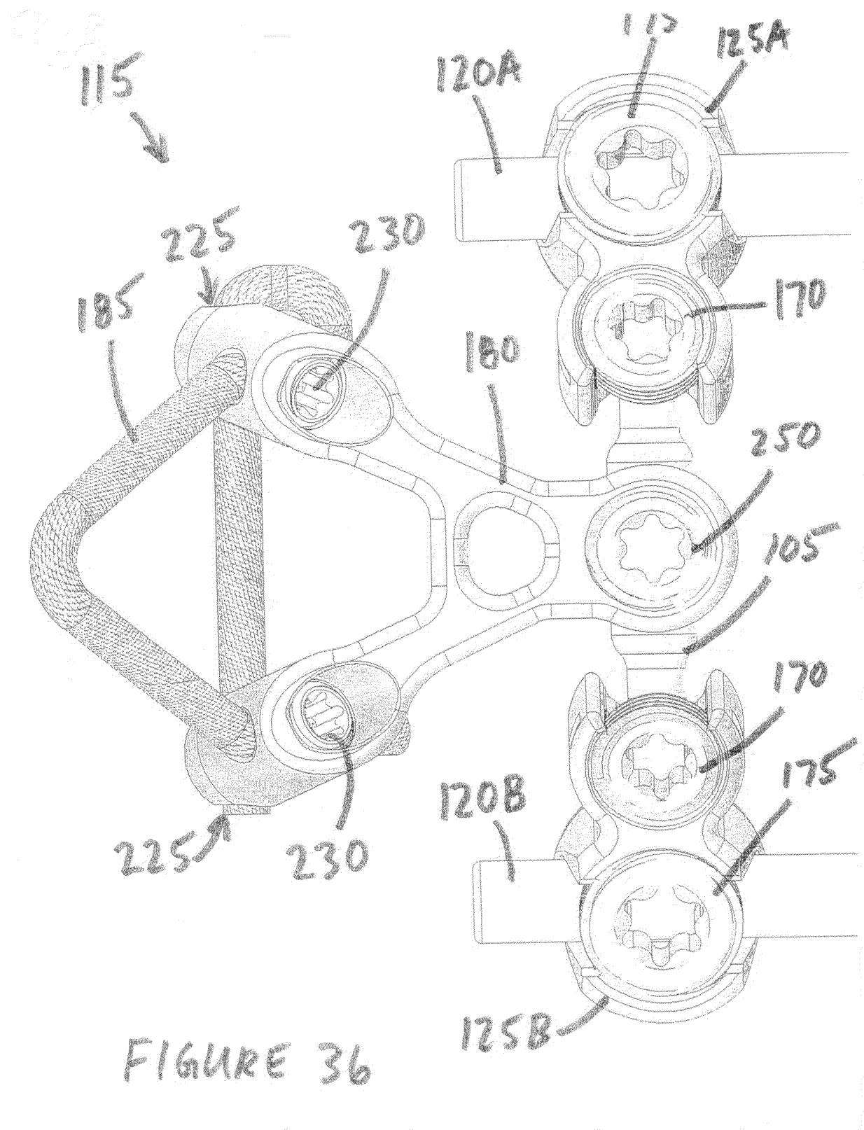

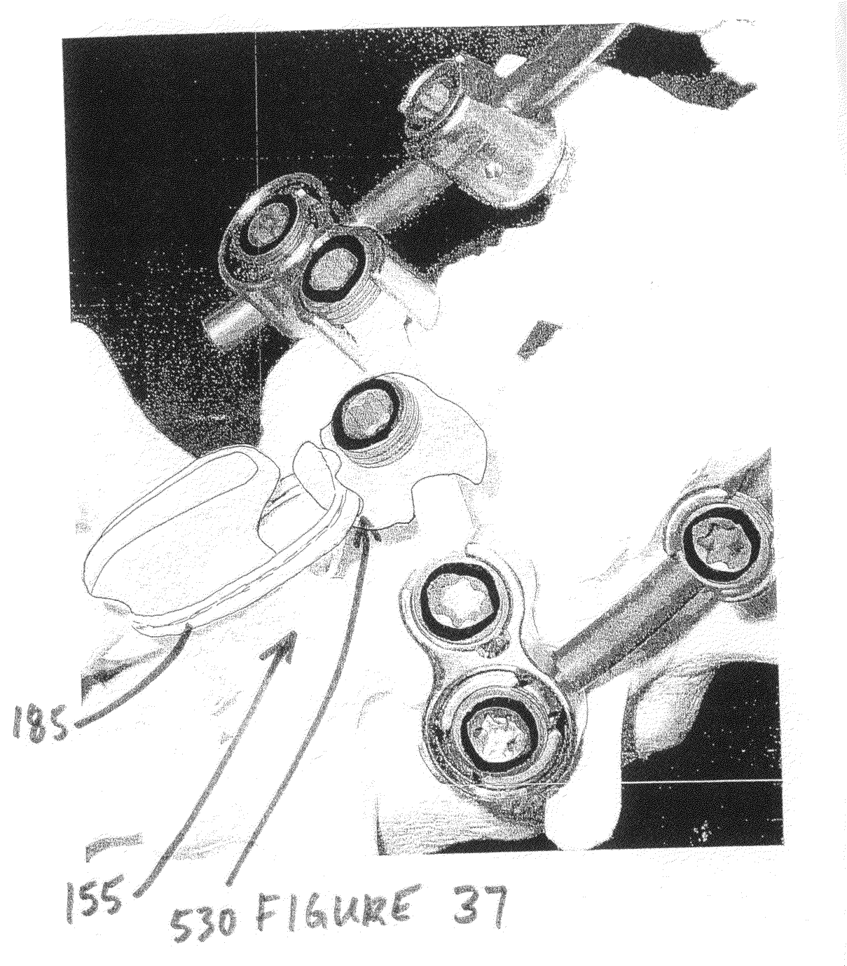

FIGS. 33-36 illustrate an embodiment of interspinous attachment device 155 with block or anchor 180 having set screws 230 to retain band 185 in respective cross-holes 225 as well as set screw 250 attaching anchor 180 to crosslink 105. FIG. 37 illustrates another embodiment of interspinous attachment device 155 having a′groove 530 to retain band 185 without or with other fixation devices. In one embodiment, band 185 may be formed having a desired length, and thereafter looped around the spinous process and into grove 530.

It will be appreciated by those skilled in the art that alternative features may be used within the scope of the present disclosure. By way of example and not limitation, various embodiments use set screws to couple or lock components together. In alternative embodiments (not shown), set screws may be replaced with alternative threaded or non-threaded fasteners such as pins, stakes, collets or other snap-in connectors, bushings, ball and detents, or the like.

What is claimed is:

1. An interspinous attachment system for providing support between adjacent spinal levels, the system comprising:
a crosslink having a first end and a second end in opposition to one another, and a distance between the first end and the second end corresponding to a distance between two spinal fixation rods fixed adjacent a spinal column of a patient;
a first crosslink connector and a second crosslink connector, each one of the first crosslink connector and the second crosslink connector having a housing with a first end and a second end in opposition to one another, the first end having a selectively attachable portion to one of the spinal fixation rods, the second end having a selectively attachable portion to one of the first end and the second end of the crosslink; and
an interspinous attachment device having a crosslink attachment portion and a spinous process attachment portion.

2. An interspinous attachment system according to claim 1, wherein the crosslink, from the first end to the second end, has a fixed length.

3. An interspinous attachment system according to claim 1, wherein the crosslink, from the first end to the second end, has a selectively variable length.

4. An interspinous attachment system according to claim 1, wherein the crosslink at the first end and the second end has a substantially spherical protuberance.

5. An interspinous attachment system according to claim 1, wherein each of the first crosslink connector and the second crosslink connector include a first set screw threadably engaging the housing at the selectively attachable portion to one of the spinal fixation rods so as to selectively retain the one of the spinal fixation rods, and a second set screw threadably engaging the housing at the selectively attachable portion to one of the first end and the second end of the crosslink so as to selectively retain the one of the first end and the second end of the crosslink.

6. An interspinous attachment system according to claim 1, wherein each of the first crosslink connector and the second crosslink connector include a single set screw threadably engaging the housing to selectively retain the one of the spinal fixation rods and the one of the first end and the second end of the crosslink.

7. An interspinous attachment system according to claim 1, wherein the crosslink attachment portion of the interspinous attachment device includes a block having a throughbore extending transversely through a central portion to receive the crosslink.

10. An interspinous attachment system according to claim 8, wherein the block comprises PEEK.

11. An interspinous attachment system according to claim 8, wherein the block comprises titanium.

12. An interspinous attachment system according to claim 8, wherein the block comprises stainless steel.

13. An interspinous attachment system according to claim 8, wherein the block comprises plastic.

14. An interspinous attachment system according to claim 7, wherein the crosslink attachment portion of the interspinous attachment device includes a fastener positionable adjacent to the throughbore extending transversely through a central portion to attach the crosslink to the crosslink attachment device.

15. An interspinous attachment system according to claim 1, wherein the spinous process attachment portion of the interspinous attachment device includes a ligament replacement band.

16. An interspinous attachment system according to claim 15, wherein the spinous process attachment portion of the interspinous attachment device includes at least one anchor point for attachment of the ligament replacement band.

17. An interspinous attachment system according to claim 15, wherein the band comprises PEEK.

18. An interspinous attachment system according to claim 15, wherein the band comprises titanium.

19. An interspinous attachment system according to claim 15, wherein the band comprises polyester.

20. An interspinous attachment system according to claim 1, wherein the spinous process attachment portion of the interspinous attachment device includes at least one set screw for attachment of ligament replacement band.

21. An interspinous attachment system according to claim 1, wherein each one of the first crosslink connector and the second crosslink connector connect directly to the crosslink, one of the spinal fixation rods, and a respective pedicle screw.

22. An interspinous attachment system according to claim 1, wherein each one of the first crosslink connector and the second crosslink connector connect directly to the crosslink and one of the spinal fixation rods.

23. An interspinous attachment system according to claim 1, wherein each one of the first crosslink connector and the second crosslink connector include a pivot between a spherical pocket formed by the housing and a rod pocket formed by the housing so as to selectively clamp the crosslink and the one of the rods, respectively.

24. An interspinous attachment system according to claim 23, wherein a pivot arm forms the rod pocket, and the rod pocket is disposed between the spherical pocket and the pivot arm.

25. An interspinous attachment system according to claim 23, wherein a swing arm within the housing forms the rod pocket, and a set screw drives the swing arm into the rod pocket to selectively attach the one of the rods to the housing.

26. An interspinous attachment system according to claim 23, wherein a pivot arm forms the rod pocket, and the pivot arm is disposed between the spherical pocket and the rod pocket.

27. A method of attaching a crosslink to a pair of spinal fixation rods, the method comprising:
providing a pair of crosslink connectors, each one of the pair of crosslink connectors having a housing with a first end and a second end in opposition to one another, the first end having a selectively attachable portion to one of the spinal fixation rods, and the second end having a selectively attachable portion to one of the first end and the second end of the crosslink;
positioning the first end and the second end of the crosslink into the second end of the crosslink connectors;
positioning the first end of each of the crosslink connectors onto one of the spinal fixation rods; and
tightening a set screw in the housing of each of the crosslink connectors to engage the first end and the second end of the crosslink in the second end of the crosslink connectors, rotate the first end of the crosslink connectors with the set screw tightening, and engage the rods with the first end of the cross link through the rotation, wherein the tightening the set screw fixedly connects both the first end and the second end of the crosslink and the rods to the housing.
